(19) United States
(12) Patent Application Publication
(10) Pub. No.: US 2023/0386668 A1
(43) Pub. Date: Nov. 30, 2023

(54) PULMONARY BLOOD FLOW CALCULATOR

(71) Applicant: Rosalba VANNI, Carate Brianza (IT)

(72) Inventor: Rosalba VANNI, Carate Brianza (IT)

(21) Appl. No.: 18/250,574

(22) PCT Filed: Oct. 26, 2021
(86) PCT No.: PCT/IB2021/059864

§ 371 Date: Apr. 26, 2023

(30) Foreign Application Priority Data
Oct. 30, 2020 (IT) 102020000025801

(51) Int. Cl. G16H 50/20; G16H 70/60

(52) U.S. Cl. CPC G16H 50/20; G16H 70/60

(57) ABSTRACT

Described herein is a method for calculating pulmonary blood flow and for using such data in the diagnosis of cardiac and pulmonary diseases, in particular, a method for diagnosing a cardiac or pulmonary disease. The method includes calculating the pulmonary blood flow (FPs) from physiological parameters measurable on a subject by a blood gas analyzer, metabolimeter, and/or cardiac output meter. Prediction equations calculated based on epidemiological data for the evaluation of FPs in a population of subjects aged between 10 and 50 years are also described. A method is also described for evaluating whether a subject undergoing cardiopulmonary exercise stress testing has actually achieved the maximum effort necessary for the correct evaluation of such a test.

### TECHNICAL FIELD OF THE INVENTION

The present invention relates to a method, in particular a computer-implemented method, for calculating pulmonary blood flow and for using such data in the diagnosis of cardiac and pulmonary diseases.

### PRIOR ART

The evaluation of parameters related to the efficiency of pulmonary function is studied for diagnostic and predictive purposes as well. Blood gas analysis currently only provides measurements of parameters such as PCO₂ and pH, calculates bicarbonate anion concentration through the Henderson-Hasselbalch equation and so on. The VCO₂ (volume of carbon dioxide exhaled per minute) is measured by a metabolic cart or system, a capnograph or a metabolimeter. Cardiac output (Qt) is measured through various types of clinical methods. However, these physiological parameters are disconnected from each other (in particular the bicarbonate in the blood and the exhaled Current knowledge affirms that only the CO₂, as such, leaves the venous blood and, crossing the alveolar epithelium by diffusion (Fick's law), reaches the alveoli and moves away from the organism with exhalation. However, these measures are applied on a physiological concept which does not reflect reality. Therapeutic choices are made on this basis, which at times turn out to be lethal for the patient.

Currently, in the clinical-diagnostic field, chronic heart failure (CHF) is determined using the NYHA (New York Heart Association) classification. It consists of four functional classes, defined in relation to the activities which the patient is able to perform. Its first publication dates back to 1964 and, although modified and perfected, it is still used for its simplicity and manageability.

This method qualitatively classifies the clinical progress of the disease in patients with chronic heart failure in relation to the degree of impairment of the usual physical activity thereof, the absolute intensity of which remains unknown, and it is used to determine a staging of the patients.

However, this modus operandi has great limitations, as it is highly subjective by both the doctor and the patient, and it generates difficulties and creates suspicion especially in the medical-legal field.

In fact, while there are no particular classification difficulties for the extreme conditions, with total absence of symptoms on one side and the presence of severe symptoms at rest on the other, for the intermediate conditions (the most frequent) the placement of a patient in Class II or III can be random and totally subjective.

Nevertheless, being the simplest to adopt, the four functional classes of the NYHA appear in the most accredited Barèmes and Tables of the law for evaluation purposes in various fields: Civil invalidity, INAIL [National Insurance Institute for Accidents at Work] tables of biological damage, private insurance, etc.

In an attempt to make the classification and consequently the staging of patients more objective, other systems have been proposed, such as the evaluation of the ejection fraction of the left ventricle, which has some advantages, but also many limitations, the cardiac index and the left ventricular end-diastolic pressure obtained at rest, but these methods are more difficult, less agile and more expensive.

Furthermore, it has previously been shown that the exercise capacity in patients with left ventricular failure is correlated with the right ventricular ejection fraction and consequently it is the pulmonary circulation that plays an important role in the degree of impairment of the patients' usual physical activity.

The World Health Organization (WHO) classifies patients with Pulmonary hypertension (PH) based on five main disease groups.

With reference to this nosographic classification, PH can complicate a left heart disease (group 2), a pulmonary disease (group 3), it can be an evolution of an unresolved thromboembolic process (group 4) or pulmonary arterial hypertension (group 1). From an epidemiological point of view, PH secondary to left heart diseases is the most frequent (78% of cases), followed by forms of PH secondary to pulmonary diseases (10% of cases) and chronic thromboembolic forms, while arterial pulmonary hypertension is the rarest form of PH (3.5% of cases) and in particular the idiopathic form.

PH is a pathophysiological condition characterized by an increase in Pulmonary Arterial Resistance (PAR), the clinical and hemodynamic effects of which are linked to the increase in afterload and the compensatory capacity of the right ventricle.

The definition of PH is hemodynamic and is established with mean pulmonary arterial pressure (PAPm) >25 mmHg at rest by right cardiac catheterization.

The definition of stress PH in the past had been established with a threshold value of 30 mmHg but is currently being reevaluated, as there are no convincing data in the literature on the hemodynamic response to exercise in healthy subjects and it has been seen that the threshold value can be widely exceeded in normal subjects.

The suspicion of PH is based on dyspnea due to effort or even at rest, asthenia and excessive fatigue with respect to daily activity.

A series of diagnostic investigations are carried out to corroborate the initial hypothesis of PH.

However, the diagnostic test which allows to confirm the PH diagnosis is right heart catheterization (diagnostic gold standard), a very invasive examination and with more or less serious possible complications up to death.

Once the clinical group of belonging has been diagnosed and identified, the NYHA classification, adapted by WHO for this disease, is also used for PH to define the functional class of patients based on the degree of impairment of the usual physical activity thereof. In fact, it is reported that the evaluation of exercise tolerance is among the best parameters for defining disease severity, monitoring the clinical course thereof and evaluating the response thereof to therapeutic treatment and is one of the best clinical indicators of prognostic stratification.

However, the problem is that PH is a devious disease which does not manifest any noticeable symptoms in the early stages thereof. In fact, 70-80% of patients are in functional class III-IV at the time of diagnosis, resulting in a very low survival percentage (from 33 months to 24 months). Therefore, early diagnosis is very important, as it allows to significantly improve the prognosis and patient quality of life.

Pulmonary thromboembolism is one of the most difficult serious diseases to recognize and diagnose, as it has unspecific symptoms which can be minimal or confused with those of other diseases (e.g., pneumonia, heart attack, asthma, etc.).

Various diagnostic tests and probability estimations (Wells score) indicative of the greater or lesser probability of having this disease are used to diagnose it.

The diagnostic test which highlights this disease with certainty is angiography with computed tomography (angiography-CT), an examination which is not without risks, although it is an improvement with respect to angiography with x-ray.

There is therefore the need to provide a new model and a method which, based on such a model, allows a better diagnosis and prognosis of a cardiac or pulmonary disease such as those outlined above and which allows correct and timely therapeutic choices.

In order to better understand the processes related to the physiology of respiration, the present inventor has verified that it is necessary to simultaneously conciliate all the chemical (law of conservation of mass or Lavoisier's law), biochemical, molecular, physiological and electrophysiological aspects related to the elimination of CO₂ from the lungs and identify the physiological parameters of interest. The theory thus elaborated gave rise to the construction of a mathematical model, the validity of which was checked through the use of experimental data present in scientific literature (Amy M. Jonk et al. 2007), estimating the parameters by comparing experimental data and theoretical results.

The present inventor has ascertained that during exhalation the diffusive flow of CO₂ is only 7% of the total CO₂ exhaled (VCO₂), while there is an important outflow of HCO₃⁻ (93% in the healthy subject at rest) and a flow of H⁺ correlated to both flows. Furthermore, the present inventor was able to calculate the pulmonary blood flow which supports the real exchange of gases, overcoming the concept of perfusion, which must take into account perfused but non-ventilated pulmonary areas, ventilated but non-perfused areas and venous-arterial shunt areas.

### SUMMARY OF THE INVENTION

The present invention relates to a method for calculating the Pulmonary Blood Flow (FPs) on the basis of physiological parameters that can be obtained in a fast and minimally invasive way through a metabolic cart or system, a capnograph or a metabolimeter, a blood gas analyzer for blood gas analysis and optionally a cardiac output meter and the use of the FPs value obtained for the diagnosis of heart and lung diseases.

An object of the present invention is therefore a method, preferably a computer-implemented method, for diagnosing a cardiac or pulmonary disease, which includes calculating pulmonary blood flow (FPs) from physiological parameters measurable on a subject by simple-to-use and low-cost instrumentation, such as blood gas analyzers, metabolimeters and cardiac output meters.

A further object of the invention is a method, preferably a computer-implemented method, for diagnosing a cardiac or pulmonary disease in a subject aged between and 50 years, which includes calculating pulmonary blood flow (FPs) based on predictive equations (obtained based on epidemiological data) applied to measurements of physiological parameters measurable with metabolimeters.

A further object of the invention is a method, preferably a computer-implemented method, for evaluating whether a subject undergoing cardiac stress testing has actually achieved the maximum effort necessary for the correct evaluation of such a test.

These and further objects, as outlined in the appended claims, will be described in the description which follows. The text of the claims must be considered included in the description for the purpose of assessing the sufficiency of the description.

For the purposes of the present invention, the term “computer” means a desktop computer, a laptop computer, a simple portable calculator or any other electronic means, programmable or non-programmable, equipped with at least one memory and at least one processor, capable of performing simple algebraic operations.

The method of the invention, in one or more of its embodiments, can be put in the form of software and loaded on a support or vector, such as, by way of non-limited example, magnetic tapes, magnetic discs, optical discs, magnetic-optical discs, ROM, PROM, VCD, DVD or other computer readable medium.

Further features and advantages of the invention will become apparent from the following description of preferred embodiments, given by way of non-limiting example.

### DETAILED DESCRIPTION OF THE INVENTION

The parameters described in the present patent application are expressed in the following dimensions:
FPs (pulmonary blood flow) in L/min
VCO₂ (exhaled CO₂ volume over time) in L/min
VCO₂/Kg in L/min·kg of body weight
Qt (cardiac output) in L/min of blood
PCO₂ᵥ (CO₂ partial pressure in venous blood) and PCO₂ₐ (CO₂ partial pressure in arterial blood) in mmHg
[HCO₃⁻]ᵥ (bicarbonate anion concentration in venous blood) and [HCO₃⁻]ₐ (bicarbonate anion concentration in arterial blood) in mmol/L
[H⁺]ᵥ (proton concentration in venous blood) and [H⁺]ₐ (proton concentration in arterial blood) in nmol/L
Φ=Greek capital letter used to define a total flow
ϕ=Greek lowercase letter used to define a partial flow
ΦCO_{2(Qt)} (total molar outflow rate in the form of CO₂ and HCO₃⁻, calculated with Qt) in mmol/min
ΦCO_{2(FPs)} (total molar outflow rate in the form of CO₂ and HCO₃⁻, calculated with FPs) in mmol/min
ΦCO₂₍ₑ₎ (total molar flow rate of exhaled CO₂) in mmol/min
ϕHCO₃⁻_{(Qt)} (molar flow, calculated with Qt, of HCO₃⁻) in mmol/min
ϕHCO₃⁻_{(FPs)} (molar flow, calculated with FPs, of HCO₃⁻) in mmol/min
ϕCO_{2(Qt)} (molar flow, calculated with Qt, of CO₂) in mmol/min
ϕCO_{2(FPs)} (molar flow, calculated with FPs, of CO₂) in mmol/min
ϕH⁺_{(FPs)} (proton outflow rate calculated with FPs) in nmol/min
ϕH⁺_{(FPs)}/FPs (proton outflow, divided by FPs) into nmol/L of pulmonary blood
ϕHCO₃⁻_{(FPs)}/FPs (bicarbonate anion outflow, divided by FPs) in mmol/L of pulmonary blood
dimensionless FPs/Qt ratio, correlated to pulmonary vascular resistance (the more FPs is lower than Qt, therefore the ratio is less than 1, the more is the pulmonary vascular resistance).

The term “about” in the present description means a variation of ±2%.

According to a first aspect, the present invention relates to a method for diagnosing a cardiac or pulmonary disease, comprising the following steps:
a) providing a set of physiological parameters of a subject at rest, wherein said parameters have been measured at a time t1, said physiological parameters being selected from exhaled CO₂ volume per minute (VCO₂), cardiac output (Qt), CO₂ partial pressure in venous blood (PCO₂ᵥ), CO₂ partial pressure in arterial blood (PCO₂ₐ), bicarbonate anion concentration in venous blood ([HCO₃⁻]ᵥ), bicarbonate anion concentration in arterial blood ([HCO₃⁻]ₐ);
b) calculating the pulmonary blood flow (FPsₜ₁) from said set of parameters of step a) according to one or both of the following algorithms:

$$FPs = ( \frac{Φ {CO}_{2 ( e )}}{Φ {CO}_{2 ( Qt )}} ) \times Qt \qquad ( A )$$

$$FPs = \frac{Φ {CO}_{2 ( e )}}{{Δ [ {HCO}_{3}^{-} ]}_{( v - a )} + {Δ [ {CO}_{2} ]}_{( v - a )}} where \qquad ( B )$$

$$Φ {CO}_{2 ( e )} = \frac{{VCO}_{2}}{22.26} ,$$

$$Φ {CO}_{2 ( Qt )} = ϕ {CO}_{2 ( Qt )} + ϕ {HCO}_{3 ( Qt )}^{-} ,$$

$$ϕ {CO}_{2 ( Qt )} = [ ( {PCO}_{2 v} - {PCO}_{2 a} ) K ] \times Qt where K = 0.03$$

$$mmol / L . mmHg and$$

$$ϕ {HCO}_{3 ( Qt )}^{-} = ( {[ {HCO}_{3}^{-} ]}_{v} - {[ {HCO}_{3}^{-} ]}_{a} ) \times Qt ,$$

$${Δ [ {HCO}_{3}^{-} ]}_{( v - a )} = {[ {HCO}_{3}^{-} ]}_{v} - {[ {HCO}_{3}^{-} ]}_{a} and$$

$${Δ [ {CO}_{2} ]}_{( v - a )} = ( {PCO}_{2 v} - {PCO}_{2 a} ) \times 0.03$$

and calculating the ratio FPSₜ₁/Qtₜ₁;
c) providing a second set of physiological parameters of a subject at rest, wherein said parameters have been measured at a time t2 subsequent to t1 by 24 hours or less, said physiological parameters being selected from exhaled CO₂ volume per minute (VCO₂), cardiac output (Qt), CO₂ partial pressure in venous blood (PCO₂ᵥ), CO₂ partial pressure in arterial blood (PCO₂ₐ), bicarbonate anion concentration in venous blood ([HCO₃⁻]ᵥ), bicarbonate anion concentration in arterial blood ([HCO₃⁻]ₐ);
d) calculating the pulmonary blood flow (FPsₜ₂) from said set of parameters of step c) according to one or both algorithms of step b) and calculating the ratio FPsₜ₂/Qtₜ₂;
e) if the ratio FPsₜ₁/Qtₜ₁ calculated in step b) is substantially equal to the ratio FPsₜ₂/Qtₜ₂ calculated in step d) and is equal to a reference value, said reference value being between 0.97 and about 0.985, and if FPsₜ₁ is substantially equal to FPsₜ₂ and therefore the ratio FPsₜ₂/FPsₜ₁ is approximately equal to 1, comparing the calculated FPs value with a reference FPs value, said reference FPs value being between 4.00 and 4.6, wherein
   a FPs/Qt value between 0.97 and 0.985 and a FPs value between 4.00 and 4.6 is indicative of a normal value or of a subject with class I chronic heart failure (CHF) (classification NYHA);
   a FPs/Qt value between 0.90 and 0.97 and a FPs value between more than 4.6 and 5.49 is indicative of a value of a subject with class II CHF (NYHA classification);
   a FPs/Qt value less than 0.90 and a FPs value greater than or equal to about 5.5 is indicative of a value of a subject with class III CHF (NYHA classification);
   a FPs/Qt value between 0.82 and 0.9 and a FPs value between 4 and 4.6 is indicative of class I pulmonary hypertension (PH) (WHO/NYHA classification);
   a FPs/Qt value between 0.60 and 0.81 and a FPs value between 3.6 and 3.99 is indicative of class II pulmonary hypertension (PH) (WHO/NYHA classification);
   a FPs/Qt value between 0.40 and 0.59 and a FPs value between 2.4 and 3.599 is indicative of class III pulmonary hypertension (PH) (WHO/NYHA classification);
   a FPs/Qt value between 0.65 and 0.85 and a FPs value of less than 2.4 is indicative of class IV pulmonary hypertension (PH) (WHO/NYHA classification) and/or class IV CHF (NYHA classification);
f) if FPsₜ₁ is substantially greater than FPsₜ₂ so that the ratio FPsₜ₂/FPsₜ₁ is less than 0.85 and FPsₜ₁/Qtₜ₁ is greater than FPsₜ₂/Qtₜ₂ and the delta thereof is between 0.585 and 0.085 and if the subject does not suffer from altitude sickness and/or high-altitude pulmonary edema (HAPE) caused by high-altitude hypoxia or does not suffer from air embolism from scuba diving with tanks and has not taken vasoconstrictive drugs, such as acetazolamide, (already established cases of vasoconstriction/obstruction, in which FPsₜ₁>FPsₜ₂), a FPsₜ₂ value substantially less than 4.00 is indicative of suspected pulmonary thromboembolism.

Pulmonary blood flow (FPs) is expressed in L/min and is calculated with algorithms A and B.

The VCO₂ (expressed in L/min) can be obtained for example by measuring with a metabolic cart or system, a capnograph or a metabolimeter.

Qt (expressed in L/min) can be measured by various methods known and reported in the literature, including Doppler echocardiography, pulse pressure methods, impedance cardiography, ultrasound dilution, electrical cardiometry, nuclear magnetic resonance and dye dilution method. If FPs (obtained with algorithm B) is greater than Qt it means that the Qt measurement is incorrect (underestimated). Therefore algorithm B immediately detects a cardiac output (Qt) measurement error.

CO₂ partial pressure in venous blood (PCO₂ᵥ) CO₂ partial pressure in arterial blood (PCO₂ₐ), bicarbonate anion concentration in venous blood ([HCO₃⁻]ᵥ), bicarbonate anion concentration in arterial blood ([HCO₃⁻]ₐ) can be obtained by blood gas analysis.

The following table I summarizes the NYHA functional classification for CHF based on the symptomatology associated with physical activity performed by the subject:

| Symptoms (fatigue, palpitations, | |
|---|---|
| dyspnea, or anginal pain) and | |
| physical activity | NYHA class |
| Asymptomatic, but with signs of | I |
| structural cardiac insult - No exercise | |
| restrictions | |
| Symptoms which appear only with | II |
| maximum effort | |
| Symptoms which already appear with | III |
| light effort | |
| Symptoms already at rest - Physical | IV |
| activity impossible | |

The following table II summarizes the functional classification of PH, made by WHO in 1998, which is based on the NYHA classification, but is adapted to this disease (indicated in the present application as the “WHO/NYHA classification”) . It is based on the symptomatology associated with physical activity performed by the subject:

| Symptoms (fatigue, dyspnea or | |
|---|---|
| asthenia, chest pain or pre- | |
| syncope) and physical activity | WHO/NYHA class |
| Asymptomatic, but with PH. | I |
| No exercise limitation | |
| Patients with PH whose symptoms | II |
| appear only with maximum effort | |
| Patients with PH whose symptoms | III |
| already appear with light effort | |
| Symptoms already at rest. | IV |
| Clinical signs of right | |
| heart failure present. | |
| Physical activity impossible | |

In preferred embodiments, the method according to the invention for diagnosing CHF comprises the following steps:
a1) providing a set of physiological parameters of a subject at rest, said physiological parameters being chosen from exhaled CO₂ volume per minute (VCO₂), cardiac output (Qt), CO₂ partial pressure in venous blood (PCO₂ᵥ), CO₂ partial pressure in arterial blood (PCO₂ₐ) bicarbonate anion concentration in venous blood ([HCO₃⁻]ᵥ), bicarbonate anion concentration in arterial blood ([HCO₃⁻]ₐ);
a2) providing a set of physiological parameters of said subject under maximum effort, said physiological parameters being chosen from exhaled CO₂ volume per minute (VO₂), cardiac output (Qt), CO₂ partial pressure in venous blood (PCO₂ᵥ), CO₂ partial pressure in arterial blood (PCO₂ₐ), bicarbonate anion concentration in venous blood ([HCO₃⁻]ₐ); arterial blood ([HCO₃⁻]ₐ);
b1) calculating the pulmonary blood flow (FPsₐ₁) from said set of parameters of step a1) and the pulmonary blood flow (FPsₐ₂) from said set of parameters of step a2) according to one or both of the following algorithms:

$$FPs = ( \frac{Φ {CO}_{2 ( e )}}{Φ {CO}_{2 ( Qt )}} ) \times Qt \qquad ( A )$$

$$FPs = \frac{Φ {CO}_{2 ( e )}}{{Δ [ {HCO}_{3}^{-} ]}_{( v - a )} + {Δ [ {CO}_{2} ]}_{( v - a )}} where \qquad ( B )$$

$$Φ {CO}_{2 ( e )} = \frac{{VCO}_{2}}{22.26}$$

$$Φ {CO}_{2 ( Qt )} = ϕ {CO}_{2 ( Qt )} + ϕ {HCO}_{3 ( Qt )}^{-} ,$$

$$ϕ {CO}_{2 ( Qt )} = [ ( {PCO}_{2 v} - {PCO}_{2 a} ) K ] \times Qt where K = 0.03$$

$$mmol / L . mmHg and$$

$$ϕ {HCO}_{3 ( Qt )}^{-} = ( {[ {HCO}_{3}^{-} ]}_{v} - {[ {HCO}_{3}^{-} ]}_{a} ) \times Qt ,$$

$${Δ [ {HCO}_{3}^{-} ]}_{( v - a )} = {[ {HCO}_{3}^{-} ]}_{v} - {[ {HCO}_{3}^{-} ]}_{a} and$$

$${Δ [ {CO}_{2} ]}_{( v - a )} = ( {PCO}_{2 v} - {PCO}_{2 a} ) \times 0.03$$

c1) comparing the values of FPsₐ₁ and FPsₐ₂ calculated with a reference FPs value, said reference FPs value being between 4.00 and 4.6, wherein
   a FPsₐ₁ value between 4.00 and 4.6 and a FPsₐ₂ value greater than 18 are indicative of a subject with class I Chronic Heart Failure (CHF) (NYHA classification);
   a FPsₐ₁ value between 4.61 and 5.49 and a FPsₐ₂ value less than 18 are indicative of a subject with class II chronic heart failure (CHF) (NYHA classification);
   a FPsₐ₁ value greater than or equal to about 5.5 and a FPsₐ₂ value less than or equal to 8.5 are indicative of a subject with class III chronic heart failure (CHF) (NYHA classification);
   a FPsₐ₁ value less than 2.4 is indicative of a subject with class IV chronic heart failure (CHF) (NYHA classification).

It should be noted that the maximum exercise effort delivered by the tested subjects can be summarized in the following table III:

| Subject NYHA class | Max exercise power (Watt) |
|---|---|
| I | ≥150 |
| II | ≤100 |
| III | ≤50 |
| IV | Stress test not executable |

In certain embodiments, the method of the invention comprises, in addition to steps a) to f) and/or steps a1) to c1), the following steps:
g) calculating the ratio

$$\frac{ϕ {HCO}_{3 ( FPs )}^{-}}{ϕ {CO}_{2 ( FPs )}}$$

$$where$$

$$ϕ {HCO}_{3 ( FPs )}^{-} = {[ {HCO}_{3}^{-} ]}_{v} - {[ {HCO}_{3}^{-} ]}_{a} \times FPs$$

$$and$$

$$ϕ {CO}_{2 ( FPs )} = [ ( {PCO}_{2 v} - {PCO}_{2 a} ) \times 0.03 ] \times FPs$$

and wherein the parameters [HCO₃⁻]ᵥ, [HCO₃⁻]ₐ, PCO₂ᵥ, PCO₂ₐ and FPs are obtained from a subject at rest;
h) assigning the value calculated according to step g) to an NYHA class for the disease CHF, wherein:
   a value greater than 12.3 and preferably less than 12.5 indicates class I (NYHA classification);
   a value between 11 and 12.3 indicates class II (NYHA classification);
   a value less than 11 and greater than 9 indicates class III (NYHA classification);
   a value between 8 and 9 indicates class IV (NYHA classification).

In a particular embodiment of the invention, the method of the present invention further comprises the following step:
i) assigning a subject suffering from CHF to class III (NYHA classification) when:
   FPs≥5.5 L/min calculated with the set of parameters of step a1);
   FPs<8.5 L/min calculated with the set of parameters of step a2);
   ΦH⁺(FPs)>18.8 nmol/min calculated with the set of parameters of step a1) with the following equation:

ΦH_{(FPs)}⁺=([H⁺]ᵥ−[H⁺]ₐ)×FPs
ΦH⁺(FPs)<154 nmol/min calculated with the set of parameters of step a2) with the following equation:

ΦH_{(FPs)}⁺=([H⁺]ᵥ−[H⁺]ₐ)×FPs;

$$\frac{ϕ {HCO}_{3 ( FPs )}^{-}}{ϕ {CO}_{2 ( FPs )}}$$

less than 11 and greater than 9 calculated with the set of parameters of step a1);

$$\frac{ϕ {HCO}_{3 ( FPs )}^{-}}{FPs} \leq 1.9 mmol / L$$

calculated with the set of parameters of step a1);
FPs/Qt<0.90 calculated with the set of parameters of step a1).

In this embodiment, the set of physiological parameters of steps a1) and a2) also included the concentration of protons in venous blood ([H⁺]ᵥ) and the concentration of protons in arterial blood ([H⁺]ₐ).

A further object of the invention is a method for evaluating a subject's achievement of a maximum effort condition during an exercise, in particular for a heart examination under effort or the like, the method comprising the following steps:
i) providing a set of physiological parameters of said subject under effort, said physiological parameters being chosen from exhaled CO₂ volume per minute (VCO₂), CO₂ partial pressure in venous blood (PCO₂ᵥ), CO₂ partial pressure in arterial blood (PCO₂ₐ), bicarbonate anion concentration in venous blood ([HCO₃⁻]ᵥ) and bicarbonate anion concentration in arterial blood ([HCO₃⁻]ₐ;
ii) calculating the pulmonary blood flow (FPs) from said set of parameters of step i) according to the following algorithm:

$$FPs = \frac{{Φ CO}_{2 ( e )}}{{Δ [ {HCO}_{3}^{-} ]}_{( v - a )} + {Δ [ {CO}_{2} ]}_{( v - a )}} \qquad ( B )$$

$$where$$

$${Φ CO}_{2 ( e )} = \frac{{VCO}_{2}}{22.26}$$

$${Δ [ {HCO}_{3}^{-} ]}_{( v - a )} = {[ {HCO}_{3}^{-} ]}_{v} - {[ {HCO}_{3}^{-} ]}_{a}$$

$${Δ [ {CO}_{2} ]}_{( v - a )} = ( {PCO}_{2 v} - {PCO}_{2 a} ) \times 0.03 ;$$

iii) calculating the ratio

$$\frac{ϕ {HCO}_{3 ( FPs )}^{-}}{ϕ {CO}_{2 ( FPs )}}$$

$$where$$

$$ϕ {HCO}_{3 ( FPs )}^{-} = ( {[ {HCO}_{3}^{-} ]}_{v} - {[ {HCO}_{3}^{-} ]}_{v} - {[ {HCO}_{3}^{-} ]}_{a} ) \times FPs$$

$$and$$

$$ϕ {CO}_{2 ( FPS )} = [ ( {PCO}_{2 v} - {PCO}_{2 a} ) \times 0.03 ] \times FPs$$

wherein the parameters [HCO₃⁻]ᵥ, [HCO₃⁻]ₐ, PCO₂ᵥ, PCO₂ₐ and FPs are obtained according to steps i) and ii);
wherein the subject has reached the maximum effort if said

$$\frac{ϕ {HCO}_{3 ( FPs )}^{-}}{ϕ {CO}_{2 ( FPs )}} max$$

ratio is ≤5.86.

The methods described above require, as mentioned, parameters which can be obtained by various instruments, including a metabolimeter (or a metabolic cart or system or a capnograph), equipment for blood gas analysis and, as regards cardiac output Qt, by complex and in some cases invasive methodologies.

Taking into account that not all analysis laboratories or clinics have the aforementioned instrumentation available, the inventor of the present patent application was able to develop prediction equations which, with excellent approximation, allow the calculation of FPs in normal and CHF class I subjects (aged 30±20 years).

The following prediction equations have been obtained by interpolation from tabulated data deriving from measurements of biochemical parameters on a population of subjects and have a high statistical significance:
C) ΦHCO₃_{_{(FPS)}}⁻=1.005·ΦCO₂₍ₑ₎^{0.9703}
R²=0.99973
R=0.9999
N=12
p-value<0.00001
D) ϕH⁺_{(FPs)}=0.4304·(ϕCO_{2(FPs)})²+11.285·ϕCO_{2(FPs)}+3.3912
R²=0.99952
R=0.9998
N=9
p-value<0.00001
   where ϕCO_{2(FPs)}=ΦCO₂₍ₑ₎−ϕHCO₃⁻_{(FPs)}
E) FPs=4.7893·ln (ϕH⁺_{(FPs)})−9.4075
R²=0.99106
R=0.9955
N=10
p-value<0.00001.

By combining equations C), D) and E) the following equation for calculating FPs can therefore be obtained:

$$FPs = 4.7893 . ln [ 0.4304 . {( Φ {CO}_{2 ( e )} - 1.005 . Φ {CO}_{2 ( e )}^{0.9703} )}^{2} + 11.285 . ( Φ {CO}_{2 ( e )} - 1.005 . Φ {CO}_{2 ( e )}^{0.9703} ) + 3.3912 ] - 9.4075 \qquad F )$$

$$where$$

$$Φ {CO}_{2 ( e )} = \frac{{VCO}_{2}}{22 , 26}$$

From the above it is clear that the pulmonary blood flow (FPs) in a subject can be calculated with high approximation by measuring the VCO₂ alone, easily obtainable by a metabolimeter, a low-cost instrument, potentially available to any laboratory or clinic, as well as to a private subject.

Therefore, according to a further aspect, the present invention relates to a diagnosis method for normal or CHF class I subjects (aged 30±20 years), comprising the following steps:
I) providing a physiological parameter of a subject at rest, wherein said parameter was measured at a time t1, said physiological parameter being the exhaled CO₂ volume per minute (VCO₂);
II) calculating the pulmonary blood flow (FPsₜ₁) from said parameter of step I) according to the following algorithm:

$$FPs = 4.7893 . ln [ 0.4304 . {( Φ {CO}_{2 ( e )} - 1.005 . Φ {CO}_{2 ( e )}^{0.9703} )}^{2} + 11.285 . ( Φ {CO}_{2 ( e )} - 1.005 . Φ {CO}_{2 ( e )}^{0.9703} ) + 3.3912 ] - 9.4075 \qquad F )$$

$$where$$

$$Φ {CO}_{2 ( e )} = \frac{{VCO}_{2}}{22.26}$$

III) providing a physiological parameter of said subject at rest, wherein said parameter was measured at a time t2 in an interval between 30 minutes and 2 hours subsequent to t1, said physiological parameter being the exhaled CO₂ volume per minute (VCO₂) and VCO₂/kg of body weight (expressed in L/min·Kg);
IV) calculating the pulmonary blood flow (FPsₜ₂) from said parameter of step III) according to the algorithm F of step II);
V) if FPsₜ₁ is substantially equal to FPsₜ₂ and therefore the ratio FPsₜ₂/FPsₜ₁ is approximately equal to 1, comparing the calculated FPs value with a reference FPs value, said reference FPs value being between 3.3 and 4.35 and a VCO₂/kg body weight value between 4.37 and 6.11 wherein
   a FPs value between 3.3 and 4.35 and a VCO₂/kg body weight value between 4.37 and 6.11 are indicative of a normal value, for subjects aged 30±20 years, or of a subject (max 50 years) with class I chronic heart failure (CHF) (NYHA classification);
   values less than 3.3 or greater than 4.35, obtained from algorithm F and a VCO₂/kg body weight value less than 4.37 or greater than 6.11, for the indicated age group, are potentially indicative of a cardiac and/or pulmonary or genetic disease (e.g., cystic fibrosis) and require confirmation by calculating the FPs with the algorithms A and/or B described above;
VI) if FPsₜ₁ is substantially greater than FPsₜ₂ so that the ratio FPsₜ₂/FPsₜ₁ is less than 0.77 and if the subject does not suffer from altitude sickness and/or high-altitude pulmonary edema (HAPE) caused by high-altitude hypoxia or does not suffer from air embolism from diving with tanks and has not taken vasoconstrictive drugs, such as acetazolamide, a FPsₜ₂ value substantially lower, preferably about 1 L/min, with respect to the value of FPsₜ₁ thereof, is indicative of suspected pulmonary thromboembolism.

As mentioned above, the simplified diagnosis method obtained by applying prediction equations is applicable to a population aged between 10 and 50 years. To avoid such a limitation, the present inventor has prepared the following simplified method which includes preparing baseline values of the parameters used for the subject.

Therefore, in a further embodiment, the simplified method according to the invention for the early detection of the onset of cardio-pulmonary diseases comprises the following steps:
I) providing a baseline physiological parameter of a negative COVID-19 subject at rest, wherein said parameter was measured at a time t1, said physiological parameter being the exhaled CO₂ volume per minute (VCO₂);
II) from the baseline VCO₂ value, obtaining FPsₜ₁ with the following algorithm (F):

$$FPs = 4.7893 . ln [ 0.4304 . {( Φ {CO}_{2 ( e )} - 1.005 . Φ {CO}_{2 ( e )}^{0.9703} )}^{2} + 11.285 . ( Φ {CO}_{2 ( e )} - 1.005 . Φ {CO}_{2 ( e )}^{0.9703} ) + 3.3912 ] - 9.4075 \qquad ( F )$$

$$where$$

$$Φ {CO}_{2 ( e )} = \frac{{VCO}_{2}}{22.26} ;$$

III) providing one or more VCO₂ values of said subject at rest, said one or more values being obtained at times tₙ subsequent to time t1;
IV) from one or more VCO₂ values of step III), obtaining respective FPsₜₙ with algorithm (F):

$$FPs = 4.7893 . ln [ 0.4304 . {( Φ {CO}_{2 ( e )} - 1.005 . Φ {CO}_{2 ( e )}^{0.9703} )}^{2} + 11.285 . ( Φ {CO}_{2 ( e )} - 1.005 . Φ {CO}_{2 ( e )}^{0.9703} ) + 3.3912 ] - 9.4075 \qquad ( F )$$

$$where$$

$$Φ {CO}_{2 ( e )} = \frac{{VCO}_{2}}{22.26} ;$$

V) comparing the one or more FPsₜₙ values according to steps III) and IV) with the respective baseline value of step I) and II) and calculating the ratio FPsₜₙ/FPsₜ₁, wherein:
   i) if there is the condition in which
   FPsₜₙ/FPsₜ₁ is greater than 1 and/or tends to increase over time from a time t2 subsequent to t1 to t-nth times subsequent to t2,
   then the subject has a probability of suffering from CHF;
   ii) if there is the condition in which
   FPsₜ₂/FPsₜ₁ is less than 1 and/or tends to decrease over time from a time t2 subsequent to t1 to t-nth times subsequent to t2,
   then the subject has a probability of suffering from undiagnosed pulmonary hypertension;
   iii) if there is the condition in which
   the ratio FPsₜ₂/FPsₜ₁ abruptly decreases from a time t2 subsequent to t1 to t-nth times subsequent to t2 by 24 hours or less and the ratio FPsₜ₂/FFsₜ₁ is less than 0.77,
   then the subject has a probability of suffering from pulmonary thromboembolism.

The repeated assessments over time as provided in steps i) and ii) are useful for monitoring any worsening of the disease and providing the competent doctor with the indications to intervene in good time.

It should be noted that the condition iii) above can also indicate a probability that the subject is infected with COVID-19, therefore the latter disease must be excluded before ascertaining the actual presence of pulmonary thromboembolism.

For those who wish to go to the mountains, one of the risks to which the body is exposed is suffering from altitude sickness and/or high-altitude pulmonary edema (HAPE) caused by hypoxia, which directly increases the resistance tone of the pulmonary vessels, causing pulmonary vasoconstriction.

Conversely, if those who carry out scuba diving with tanks do not ascend to the surface with the necessary precautions, they may be subject to the formation of gaseous micro-emboli and therefore to a concrete risk of death.

The method using the prediction equations described above can therefore provide a valid tool for monitoring the onset of the aforesaid problems and therefore being able to intervene in good time.

For obvious practical reasons, VCO₂ monitoring over time is more easily achievable in the case of high-altitude climbing, while in the case of scuba diving, a VCO₂ analysis system should be included integrated with diving equipment, for example by a metabolimeter with wireless transmission of the detected data, so that a diver's assistant on the surface can constantly monitor the parameters and provide real-time indications to the diver.

Therefore, a further embodiment of the invention is a method for monitoring the pulmonary parameters of a subject practicing high-altitude climbing or scuba diving with a tank, comprising the following steps:
I) providing a baseline physiological parameter of a subject at rest, wherein said parameter was measured at a time t1, said physiological parameter being the exhaled CO₂ volume per minute (VCO₂);
II) from the baseline VCO₂ value, obtaining FPsₜ₁ with the following algorithm (F):

$$FPs = 4.7893 . ln [ 0.4304 . {( Φ {CO}_{2 ( e )} - 1.005 . Φ {CO}_{2 ( e )}^{0.9703} )}^{2} + 11.285 . ( Φ {CO}_{2 ( e )} - 1.005 . Φ {CO}_{2 ( e )}^{0.9703} ) + 3.3912 ] - 9.4075 \qquad ( F )$$

$$where$$

$$Φ {CO}_{2 ( e )} = \frac{{VCO}_{2}}{22.26} ;$$

III) providing one or more VCO₂ values of said subject at rest, said one or more values being obtained at times tₙ subsequent to time t1;
IV) from one or more VCO₂ values of step III), obtaining respective FPsₜₙ with algorithm (F):

$$FPs = 4.7893 . ln [ 0.4304 . {( Φ {CO}_{2 ( e )} - 1.005 . Φ {CO}_{2 ( e )}^{0.9703} )}^{2} + 11.285 . ( Φ {CO}_{2 ( e )} - 1.005 . Φ {CO}_{2 ( e )}^{0.9703} ) + 3.3912 ] - 9.4075$$

$$where$$

$$Φ {CO}_{2 ( e )} = \frac{{VCO}_{2}}{22.26} ;$$

V) comparing the one or more FPsₜₙ values according to steps III) and IV) with the respective baseline value of step I) and II) and calculating the ratio FPsₜₙ/FPsₜ₁, is wherein:
if there is the condition in which
   the FPsₜₙ value compared to the baseline FPsₜ₁ decreased by about 1 unit and the ratio FPsₜₙ/FPsₜ₁ is between 0.75 and 0.80,
then the subject has developed a risk of acute cardio-pulmonary disease.

In particular, for those who practice high-altitude climbing, the acute cardio-pulmonary disease is altitude sickness and/or, with the permanence at that altitude, a high-altitude pulmonary edema (HAPE). In this case, a rapid descent to a lower altitude is recommended.

For those who practice scuba diving with a tank, the cardio-pulmonary disease is an air embolism. In this case, the ascent to the surface must be interrupted or slowed down and the subject must then be treated with oxygen and/or in a hyperbaric chamber.

It should be noted that the Qt value determined with the known methods described above is not always correct. In fact, it may be underestimated, with the clinical consequences which can be imagined.

The FPs value calculated as described above allows to verify the correctness of the Qt value. Indeed, it is impossible for Qt to be less than FPs. Hence, when the value of Qt is less than the value of FPs, it means that Qt has been underestimated.

Therefore, a further object of the invention is a method for checking the validity of cardiac output (Qt) measurements and immediate detection of a measurement error, comprising a step of comparing the Qt measurement value with the calculated Pulmonary Blood Flow (FPs) value, obtained with the algorithm B

$$FPs = \frac{{Φ CO}_{2 ( e )}}{{Δ [ {HCO}_{3}^{-} ]}_{( v - a )} + {Δ [ {CO}_{2} ]}_{( v - a )}}$$

$$where$$

$${Φ CO}_{2 ( e )} = \frac{{VCO}_{2}}{22.26} ,$$

$${Δ [ {HCO}_{3}^{-} ]}_{( v - a )} = {[ {HCO}_{3}^{-} ]}_{v} - {[ {HCO}_{3}^{-} ]}_{a} and$$

$${Δ [ {CO}_{2} ]}_{( v - a )} = ( {PCO}_{2 v} - {PCO}_{2 a} ) \times 0.03 ,$$

comprising the following steps:
   1) calculating FPs with said algorithm B
   2) comparing the Qt value with the FPs value:
if Qt is greater than FPs, the Qt measurement is correct
if Qt is less than FPs, the Qt measurement is incorrect (underestimated).

All the methods described above in detail can be achieved with the aid of a computer and can be made into a software that can be run on said computer. The term “computer”, as previously defined, must be understood in a broad sense, thus also including a common portable calculator as an aid for calculating the values to be entered in the equations defined above.

From the above it is clear that the method of the present invention at least partially achieves the intended objects, since:
it allows an objective classification of the four NYHA classes for CHF and the four WHO/NYHA classes for PH;
it allows an objective staging of class II or III CHF patients (the most frequent) and an objective staging of PH patients;
it allows a reduction in the diagnostic path for PH, a reduction in the number of false positives for PH after the preliminary diagnostic path, with consequent. savings in terms of performing right cardiac catheterization which is invasive for the patient and expensive for the healthcare system;
it allows an early identification of subjects with probable PH, through periodic mass screening of risk groups, thus solving the serious problem of late diagnosis; the risk groups are those listed below (derived from the WHO Pulmonary Hypertension Classification):
1. Arterial pulmonary hypertension:
1A Familial and Heritable
1B Associated with:
1B.1 Connectivitis (e.g., systemic lupus erythematosus, scleroderma, rheumatoid arthritis, Sjögren's syndrome, undifferentiated connective tissue diseases, dermatomyositis)
1B.2 Systemic-pulmonary shunt
1B.3 Portal hypertension
1B.4 HIV infection
1B.5 Drugs (e.g., amphetamines, methamphetamines) and toxic substances
1B.6 Other (thyroid disease, glycogen storage disease, Gaucher's disease, hereditary hemorrhagic telangiectasia, hemoglobinopathies, myeloproliferative diseases, splenectomy)
1C Associated with significant venous or capillary involvement:
1C.1 Veno-occlusive pulmonary disease
1C.2 Pulmonary capillary hemangiomatosis
1D Persistent pulmonary hypertension in the newborn
2. Hypertension associated with left heart disease
2A Disease of the atrium or left ventricle
2B Valvular disease of the left heart
3. Pulmonary hypertension associated with pulmonary disease and/or hypoxemia
3A Chronic obstructive pulmonary disease
3B Interstitial lung disease
3C Breathing disorders during sleep
3D Disorders associated with alveolar hypoventilation
3E Chronic exposure to high altitude
3F Congenital abnormalities
4. Pulmonary hypertension secondary to chronic thromboembolic diseases
4A Thromboembolic obstruction of proximal pulmonary arteries
4B Thromboembolic obstruction of distal pulmonary arteries
4C Non-thrombotic pulmonary embolisms (tumors, parasites, foreign bodies).
5. Miscellaneous

Sarcoidosis, Histiocytosis X, lymphangiomatosis, compression of the pulmonary vessels from adenopathy, tumor, fibrosing mediastinitis or other process;
it allows an early detection of secondary PH in groups 2 and 3 and in the presence of associated diseases such as scleroderma (PH is the most fearful complication for this disease);
it allows an immediate indication of the presence of an obstruction of the pulmonary blood flow (presumably caused by thrombi/emboli) through the verification of a sudden decrease in pulmonary blood flow; however, it is first necessary to ascertain and exclude whether pulmonary vasoconstrictor drugs have been used (e.g., acetazolamide) or the presence of a Covid-19 infection (with a swab);
it allows an assessment of the severity of the disease in progress (due to the size of the thrombus/embolus and the extent of the obstructed vascular bed) depending on the more or less conspicuous decrease in pulmonary blood flow and the FPs/Qt ratio (correlated to pulmonary vascular resistance); this evaluation can also be used for subjects who have gone scuba diving with tanks (for suspected air embolism) or high-altitude hypoxia (in the presence of altitude sickness and/or pulmonary edema (HAPE);
it allows public health surveillance (for pulmonary thromboembolism, pulmonary hypertension with groups at risk of PH and CHF) as a method (with algorithm F) applied to widely used instruments for domestic use;
it allows an objective assessment of the degree of impairment of physical activity both at rest and during exercise;
it allows an objective assessment of the maximum exercise intensity;
it allows an evaluation of pulmonary circulation through the quantification of pulmonary blood flow (FPs) and the FPs/Qt ratio (correlated to pulmonary vascular resistance);
it allows monitoring the clinical course and evaluating the response to therapeutic treatment;
it allows a verification of the possible presence of pulmonary hypertension secondary to left heart diseases;
it benefits from very high reliability;
it is not very invasive;
it is inexpensive.

What we claim is:

1. A method for diagnosing a cardiac or pulmonary disease comprising the following steps:
a) providing a set of physiological parameters of a subject at rest, wherein said parameters have been measured at a time t1, said physiological parameters being selected from exhaled CO₂ volume per minute (VCO₂), cardiac output (Qt), CO₂ partial pressure in venous blood (PCO₂ᵥ), CO₂ partial pressure in arterial blood (PCO₂ₐ), bicarbonate anion concentration in venous blood ([HCO₃⁻]ᵥ), and bicarbonate anion concentration in arterial blood ([HCO₃⁻]ₐ);
b) calculating the pulmonary blood flow (FPsₜ₁) from said set of parameters of step a) according to one or both of the following algorithms:
$$FPs = ( \frac{Φ {CO}_{2 ( e )}}{Φ {CO}_{2 ( Qt )}} ) \times Qt \qquad ( A )$$
$$FPs = \frac{Φ {CO}_{2 ( e )}}{{Δ [ {HCO}_{3}^{-} ]}_{( v - a )} + {Δ [ {CO}_{2} ]}_{( v - a )}} \qquad ( B )$$
$$where$$
$$Φ {CO}_{2 ( e )} = \frac{{VCO}_{2}}{22.26} ,$$
$$Φ {CO}_{2 ( Qt )} = ϕ {CO}_{2 ( Qt )} + ϕ {HCO}_{3 ( Qt )}^{-}$$
$$ϕ {CO}_{2 ( Qt )} = [ ( {PCO}_{2 v} - {PCO}_{2 a} ) K ] \times Qt where K = 0.03 mmol / L . mmHg and$$
$$ϕ {HCO}_{3 ( Qt )}^{-} = ( {[ {HCO}_{3}^{-} ]}_{v} - {[ {HCO}_{3}^{-} ]}_{a} ) \times Qt ,$$
$${Δ [ {HCO}_{3}^{-} ]}_{( v - a )} = {[ {HCO}_{3}^{-} ]}_{v} - {[ {HCO}_{3}^{-} ]}_{a} and$$
$${Δ [ {CO}_{2} ]}_{( v - a )} = ( {PCO}_{2 v} - {PCO}_{2 a} ) \times 0.03$$
and
calculating the ratio FPsₜ₁/Qtₜ₁:
c) providing a second set of physiological parameters of a subject at rest, wherein said parameters have been measured at a time t2 subsequent to t1 by 24 hours or less, said physiological parameters being selected from exhaled CO₂ volume per minute (VCO₂), cardiac output (Qt), CO₂ partial pressure in venous blood (PCO₂ᵥ), CO₂ partial pressure in arterial blood (PCO₂ₐ), bicarbonate anion concentration in venous blood ([HCO₃⁻]ᵥ), and bicarbonate anion concentration in arterial blood ([HCO₃⁻]ₐ);
d) calculating the pulmonary blood flow (FPsₜ₂) from said set of parameters of step c) according to one or both algorithms of step b) and calculating the ratio FPsₜ₂/Qtₜ₂;
e) if the ratio FPsₜ₁/Qtₜ₁ calculated in step b) is substantially equal to the ratio FPsₜ₂/Qtₜ₂ calculated in step d) and is equal to a reference value, said reference value being between 0.97 and 0.985, and if FPsₜ₁ is substantially equal to FPsₜ₂, and therefore the ratio FPsₜ₂/FPsₜ₁ is approximately equal to 1, comparing the calculated FPs value with a reference FPs value, said reference FPs value being between 4.00 and 4.6, wherein
a FPs/Qt value between 0.97 and 0.985 and a FPs value between 4.00 and 4.6 is indicative of a normal value or of a subject with class I chronic heart failure (CHF) (classification NYHA);
a FPs/Qt value between 0.90 and 0.977 and a FPs value between more than 4.6 and 5.49 is indicative of a value of a subject with class II CHF (NYHA classification);
a FPs/Qt value less than 0.90 and a FPs value greater than or equal to about 5.5 is indicative of a value of a subject with class Ill CHF (NYHA classification);
a FPs/Qt value between 0.82 and 0.9 and a FPs value between 4 and 4.6 is indicative of class I pulmonary hypertension (PH) (WHO/NYHA classification);
a FPs/Qt value between 0.60 and 0.81 and a FPs value between 3.6 and 3.99 is indicative of class II pulmonary hypertension (PH) (WHO/NYHA classification);
a FPs/Qt value between 0.40 and 0.59 and a FPs value between 2.4 and 3.599 is indicative of class III pulmonary hypertension (PH) (WHO/NYHA classification); and
a FPs/Qt value between 0.65 and 0.85 and a FPs value of less than 2.4 is indicative of class IV pulmonary hypertension (PH) (WHO/NYHA classification) and/or class IV CHF (NYHA classification);
f) if FPsₜ₁ is substantially greater than FPsₜ₂ so that the ratio FPsₜ₂/FPsₜ₁ is less than 0.85 and FPSₜ₁/Qtₜ₁ is greater than FPsₜ₂/Qtₜ₂ and the delta thereof is between 0.585 and 0.085 and if the subject does not suffer from altitude sickness and/or high-altitude pulmonary edema (HAPE) caused by high-altitude hypoxia or does not suffer from air embolism from scuba diving with tanks and has not taken vasoconstrictive drugs, such as acetazolamide, a FPsₜ₂ value substantially less than 4.00 is indicative of suspected pulmonary thromboembolism,
wherein this method is optionally implemented by computer.

2. The method of diagnosing PH according to claim 1, wherein the functional PH classification, which is based on the WHO/NYHA classification, based on the symptoms associated with physical activity performed by the subject, is shown in the following table:

| Symptoms (fatigue, dyspnea or | |
|---|---|
| asthenia, chest pain or pre-syncope) | |
| and physical activity | WHO/NYHA class |
| Asymptomatic, but with PH. No | I |
| exercise limitation | |
| Patients with PH whose symptoms | II |
| appear only with maximum effort | |
| Patients with PH whose symptoms | III |
| already appear with light effort | |
| Symptoms already at rest. Clinical | IV |
| signs of right heart failure present. | |
| Physical activity impossible | |

3. A method of diagnosing CHF, comprising the following steps:
a1) providing a set of physiological parameters of a subject at rest, said physiological parameters being chosen from exhaled CO₂ volume per minute (VCO₂), cardiac output (Qt), CO₂ partial pressure in venous blood (PCO₂ᵥ), CO₂ partial pressure in arterial blood (PCO₂ₐ), bicarbonate anion concentration in venous blood ([HCO₃⁻]ᵥ), and bicarbonate anion concentration in arterial blood ([HCO₃⁻]ₐ);
a2) providing a set of physiological parameters of said subject under maximum effort, said physiological parameters being chosen from exhaled CO₂ volume per minute (VCO₂), cardiac output (Qt), CO₂ partial pressure in venous blood (PCO₂ᵥ), CO₂ partial pressure in arterial blood (PCO₂ₐ), bicarbonate anion concentration in venous blood ([HCO₃⁻]ᵥ), and bicarbonate anion concentration in arterial blood ([HCO₃⁻]ₐ);
b1) calculating the pulmonary blood flow (FPsₐ₁) from said set of parameters of step a1) and the pulmonary blood flow (FPsₐ₂) from said set of parameters of step a2) according to one or both of the following algorithms:
$$FPs = ( \frac{Φ {CO}_{2 ( e )}}{Φ {CO}_{2 ( Qt )}} ) \times Qt \qquad ( A )$$
$$FPs = \frac{Φ {CO}_{2 ( e )}}{{Δ [ {HCO}_{3}^{-} ]}_{( v - a )} + {Δ [ {CO}_{2} ]}_{( v - a )}} \qquad ( B )$$
$$where$$
$$Φ {CO}_{2 ( e )} = \frac{{VCO}_{2}}{22.26} ,$$
$$Φ {CO}_{2 ( Qt )} = ϕ {CO}_{2 ( Qt )} + ϕ {HCO}_{3 ( Qt )}^{-}$$
$$ϕ {CO}_{2 ( Qt )} = [ ( {PCO}_{2 v} - {PCO}_{2 a} ) K ] \times Qt where K = 0.03 mmol / L . mmHg and$$
$$ϕ {HCO}_{3 ( Qt )}^{-} = ( {[ {HCO}_{3}^{-} ]}_{v} - {[ {HCO}_{3}^{-} ]}_{a} ) \times Qt ,$$
$${Δ [ {HCO}_{3}^{-} ]}_{( v - a )} = {[ {HCO}_{3}^{-} ]}_{v} - {[ {HCO}_{3}^{-} ]}_{a} and$$
$${Δ [ {CO}_{2} ]}_{( v - a )} = ( {PCO}_{2 v} - {PCO}_{2 a} ) \times 0.03$$
c1) comparing the values of FPsₐ₁ and FPsₐ₂ calculated with a reference FPs value, said reference FPs value being between 4.00 and 4.6, wherein
a FPsₐ₁ value between 4.00 and 4.6 and a FPsₐ₂ value greater than 18 are indicative of a subject with class I Chronic Heart Failure (CHF) (NYHA classification);
a FPsₐ₁ value between 4.61 and 5.49 and a FPsₐ₂ value less than 18 are indicative of a subject with class II chronic heart failure (CHF) (NYHA classification);
a FPsₐ₁ value greater than or equal to 5.5 and a FPsₐ₂ value less than or equal to 8.5 are indicative of a subject with class III chronic heart failure (CHF) (NYHA classification); and
a FPsₐ₁ value less than 2.4 is indicative of a subject with class IV chronic heart failure (CHF) (NYHA classification),
wherein this method is optionally implemented by computer.

4. The method according to claim 1, wherein the NYHA functional classification for CHF based on the symptomatology associated with physical activity performed by the subject is shown in the following table:

| Symptoms (fatigue, palpitations, | |
|---|---|
| dyspnea, or anginal pain) and | |
| physical activity | NYHA class: |
| Asymptomatic, but with signs of | I |
| structural cardiac insult - No exercise | |
| restrictions | |
| Symptoms which appear only with | II |
| maximum effort | |
| Symptoms which already appear with | III |
| light effort | |
| Symptoms already at rest - Physical | IV |
| activity impossible | |

5. The method according to claim 3, wherein the maximum exercise power delivered by the tested subjects is shown in the following table:

| Subject NYHA class | Max exercise power (Watt) |
|---|---|
| I | ≥150 |
| II | ≤100 |
| III | ≤50 |
| IV | Stress test not executable |

6. The method according to claim 1, further comprising the following steps:
g) calculating the ratio
$$\frac{{{ΦHCO}_{3}^{-}}_{( FPs )}}{{ΦCO}_{2 ( FPS )}} where {{ΦHCO}_{3}^{-}}_{( FPs ) = {[ {HCO}_{3}^{-} ]}_{v} - {[ {HCO}_{3}^{-} ]}_{a} \times FPs}$$
$$and {{ΦCO}_{2}}_{( FPs ) = [ ( {PCO}_{2 v} - {PCO}_{2 a} ) \times 0.03 \times FPs}$$
and wherein the parameters [HCO₃⁻]ᵥ, [HCO₃⁻]ₐ, PCO₂ᵥ, PCO₂ₐ and FPs are obtained from a subject at rest;
h) assigning the value calculated according to step g) to an NYHA class for the disease CHF, wherein:
a value greater than 12.3 and preferably less than 12.5 indicates class I (NYHA classification);
a value between 11 and 12.3 indicates class II (NYHA classification);
a value less than 11 and greater than 9 indicates class III (NYHA classification); and
a value between 8 and 9 indicates class IV (NYHA classification).

7. The method according to claim 1, wherein the set of physiological parameters of steps a1) and a2) further comprises the proton concentration in venous blood ([H⁺]ᵥ) and the proton concentration in arterial blood ([H⁺]ₐ), the method comprising the following step:
i) assigning a subject suffering from CHF to class III (NYHA classification) when:
FPs≥5.5 L/min calculated with the set of parameters of step a1);
FPs<8.5 L/min calculated with the set of parameters of step a2);
ΦH⁺FPs)>18.8 nmol/min calculated with the set of parameters of step a1) with the following equation:
ΦH_{(FPs)}⁺=([H⁺]ᵥ−[H⁺]ₐ)×FPs
ΦH⁺(FPs)<154 nmol/min calculated with the set of parameters of step a2) with the following equation:
ΦH_{(FPs)}⁺=([H⁺]ᵥ−[H⁺]ₐ)×FPs;
$$\frac{{{ΦHCO}_{3}^{-}}_{( FPs )}}{{{ΦCO}_{2}}_{( FPs )}}$$
less than 11 and greater than 9 calculated with the set of parameters of step a1);
$$\frac{{{ΦHCO}_{3}^{-}}_{( FPs )}}{FPs} \leq 1.9 mmol / L$$
calculated with the set of parameters of step a1);
FPs/Qt<0.90 calculated with the set of parameters of step a1).

8. A diagnosis method for normal or class I CHF subjects in a population aged 10 to 50, comprising the following steps:
I) providing a physiological parameter of a subject at rest, wherein said parameter was measured at a time t1, said physiological parameter being the exhaled CO₂ volume per minute (VCO₂);
II) calculating the pulmonary blood flow (FPsₜ₁) from said parameter of step I) according to the following algorithm:
$$F ) FPs = 4.7893 . ln [ 0.4304 . {( {ΦCO}_{2 ( e )} - 1.005 . {ΦCO}_{2 ( e )}^{0.9703} )}^{2} + 11.285 . ( {ΦCO}_{2 ( e )} - 1.005 . {ΦCO}_{2 ( e )}^{0.9703} ) + 3.3912 ] - 9.4075 where$$
$${ΦCO}_{2 ( e )} = \frac{{VCO}_{2}}{22.26}$$
III) providing a physiological parameter of said subject at rest, wherein said parameter was measured at a time t2 in an interval between 30 minutes and 2 hours subsequent to t1, said physiological parameter being the exhaled CO₂ volume per minute (VCO₂) and VCO₂/kg of body weight (expressed in L/min·Kg);
IV) calculating the pulmonary blood flow (FPsₜ₂) from said parameter of step III) according to the algorithm F of step II);
V) if FPsₜ₁ is substantially equal to FPsₜ₂ and therefore the ratio FPsₜ₂/FPsₜ₁ is approximately equal to 1, comparing the calculated FPs value with a reference FPs value, said reference FPs value being between 3.3 and 4.35 and a VCO₂/kg body weight value between 4.37 and 6.11 wherein
a FPs value between 3.3 and 4.35 and a VCO₂/kg body weight value between 4.37 and 6.11 are indicative of a normal value, for subjects aged between 10 and 50, or of a subject (max 50 years) with class I chronic heart failure (CHF) (NYHA classification); and
values less than 3.3 or greater than 4.35, obtained from algorithm F and a VCO₂/kg body weight value less than 4.37 or greater than 6.11, for the indicated age group, are potentially indicative of a cardiac and/or pulmonary or genetic disease (e.g., cystic fibrosis) and require confirmation by calculating the FPs with the algorithms A and/or B described in claim 1, step b) and in claim 3, step b1); and
VI) if FPsₜ₁ is substantially greater than FPsₜ₂ so that the ratio FPsₜ₂/FPsₜ₁ is less than 0.77 and if the subject does not suffer from altitude sickness and/or high-altitude pulmonary edema (HAPE) caused by high-altitude hypoxia or does not suffer from air embolism from diving with tanks and has not taken vasoconstrictive drugs, such as acetazolamide, a FPsₜ₂ value substantially lower with respect to the value of FPsₜ₁ thereof, preferably about 1 L/min, is indicative of suspected pulmonary thromboembolism,
wherein this method is optionally implemented by computer.

9. A method for evaluating a subject's achievement of a maximum effort condition during an exercise, in particular for a heart examination under effort or the like, the method comprising the following steps:
i) providing a set of physiological parameters of said subject under effort, said physiological parameters being chosen from exhaled CO₂ volume per minute (VCO₂), CO₂ partial pressure in venous blood (PCO₂), CO₂ partial pressure in arterial blood (PCO₂ₐ), bicarbonate anion concentration in venous blood ([HCO₃⁻]ᵥ), and bicarbonate anion concentration in arterial blood ([HCO₃⁻]ₐ; and
ii) calculating the pulmonary blood flow (FPs) from said set of parameters of step i) according to the following algorithm:
$$FPs = \frac{{ΦCO}_{2 ( e )}}{{Δ [ {HCO}_{3}^{-} ]}_{( v - a )} + {Δ [ {CO}_{3} ]}_{( v - a )}} \qquad ( B )$$
$$where {ΦCO}_{2 ( e )} = \frac{{VCO}_{2}}{22.26}$$
$${Δ [ {HCO}_{3}^{-} ]}_{( v - a )} = {[ {HCO}_{3}^{-} ]}_{v} - {[ {HCO}_{3}^{-} ]}_{a}$$
$${Δ [ {CO}_{2} ]}_{( v - a )} = ( {PCO}_{2 v} - {PCO}_{2 a} ) \times 0.03 ;$$
iii) calculating the ratio
$$\frac{{{ΦHCO}_{3}^{-}}_{( FPs )}}{{ΦCO}_{2 ( FPS )}} where {{ΦHCO}_{3}^{-}}_{( FPs ) = {[ {HCO}_{3}^{-} ]}_{v} - {[ {HCO}_{3}^{-} ]}_{a} \times FPs}$$
$$and {{ΦCO}_{2}}_{( FPs ) = [ ( {PCO}_{2 v} - {PCO}_{2 a} ) \times 0.03 \times FPs}$$
wherein the parameters [HCO₃⁻]ᵥ, [HCO₃⁻]ₐ, PCO₂ᵥ, PCO₂ₐ and FPs are obtained according to steps i) and ii);
wherein the subject has reached the maximum effort if said
$$\frac{{{ΦHCO}_{3}^{-}}_{( FPs )}}{{ΦCO}_{2 ( FPS )}} max ratio is \leq 5.86$$
wherein this method is optionally implemented by computer.

10. A method for checking the validity of cardiac output (Qt) measurements and immediate detection of a measurement error, comprising a step of comparing the Qt measurement value with the calculated Pulmonary Blood Flow (FPs) value, obtained with the algorithm B
$$FPs = \frac{{ΦCO}_{2 ( e )}}{{Δ [ {HCO}_{3}^{-} ]}_{( v - a )} + {Δ [ {CO}_{3} ]}_{( v - a )}} \qquad ( B )$$
$$where {ΦCO}_{2 ( e )} = \frac{{VCO}_{2}}{22 , 26} ,$$
$${Δ [ {HCO}_{3}^{-} ]}_{( v - a )} = {[ {HCO}_{3}^{-} ]}_{v} - {[ {HCO}_{3}^{-} ]}_{a} and$$
$${Δ [ {CO}_{2} ]}_{( v - a )} = ( {PCO}_{2 v} - {PCO}_{2 a} ) \times 0.03 ,$$
comprising the following steps:
1) calculating FPs with said algorithm B; and
2) comparing the Qt value with the FPs value:
if Qt is greater than FPs, the Qt measurement is correct, and
if Qt is less than FPs, the Qt measurement is incorrect (underestimated),
wherein this method is optionally implemented by computer.

11. A simplified method of detecting early the onset of cardio-pulmonary diseases, comprising the following steps:
I) providing a baseline physiological parameter of a negative COVID-19 subject at rest, wherein said parameter was measured at a time t1, said physiological parameter being the exhaled CO₂ volume per minute (VCO₂);
II) from the baseline VCO₂ value, obtaining FPsₜ₁ with the following algorithm (F):
$$FPs = 4.7893 . ln [ 0.4304 . {( {ΦCO}_{2 ( e )} - 1.005 . {ΦCO}_{2 ( e )}^{0.9703} )}^{2} + 11.285 . ( {ΦCO}_{2 ( e )} - 1.005 . {ΦCO}_{2 ( e )}^{0.9703} ) + 3.3912 ] - 9.4075 \qquad ( F )$$
$$where {ΦCO}_{2 ( e )} = \frac{{VCO}_{2}}{22.26}$$
III) providing one or more VCO₂ values of said subject at rest, said one or more values being obtained at times t o subsequent to time t1;
IV) from one or more VCO₂ values of step III), obtaining respective FPsₜₙ with algorithm (F):
$$FPs = 4.7893 . ln [ 0.4304 . {( {ΦCO}_{2 ( e )} - 1.005 . {ΦCO}_{2 ( e )}^{0.9703} )}^{2} + 11.285 . ( {ΦCO}_{2 ( e )} - 1.005 . {ΦCO}_{2 ( e )}^{0.9703} ) + 3.3912 ] - 9.4075 \qquad ( F )$$
$$where {ΦCO}_{2 ( e )} = \frac{{VCO}_{2}}{22.26}$$
and
V) comparing the one or more FPsₜₙ values according to steps III) and IV) with the respective baseline value of step I) and II) and calculating the ratio FPsₜₙ/FPsₜ₁, wherein:
i) if there is the condition in which
FPsₜₙ/FPsₜ₁ is greater than 1 and/or tends to increase over time from a time t2 subsequent to t1 to t-nth times subsequent to t2,
then the subject has a probability of suffering from CHF;
ii) if there is the condition in which
FPsₜ₂/FPsₜ₁ is less than 1 and/or tends to decrease over time from a time t2 subsequent to t1 to t-nth times subsequent to t2,
then the subject has a probability of suffering from pulmonary hypertension not yet diagnosed; and
iii) if there is the condition in which
the ratio FPsₜ₁/FPsₜ₁ abruptly decreases from a time t2 subsequent to t1 to t-nth times subsequent to t2 by 24 hours or less and the ratio FPsₜ₂/FPsₜ₁ is less than 0.77, then the subject has a probability of suffering from pulmonary thromboembolism,
wherein this method is optionally implemented by computer.

12. A method of monitoring the pulmonary parameters of a subject practicing high-altitude climbing or scuba diving with a tank, comprising the following steps:
I) providing a baseline physiological parameter of a subject at rest, wherein said parameter was measured at a time t1, said physiological parameter being the exhaled CO₂ volume per minute (VCO₂);
II) from the baseline VCO₂ value, obtaining FPsₜ₁ with the following algorithm (F):
$$FPs = 4.7893 . ln [ 0.4304 . {( {ΦCO}_{2 ( e )} - 1.005 . {ΦCO}_{2 ( e )}^{0.9703} )}^{2} + 11.285 . ( {ΦCO}_{2 ( e )} - 1.005 . {ΦCO}_{2 ( e )}^{0.9703} ) + 3.3912 ] - 9.4075 \qquad ( F )$$
$$where {ΦCO}_{2 ( e )} = \frac{{VCO}_{2}}{22.26}$$
III) providing one or more VCO₂ values of said subject at rest, said one or more values being obtained at times tₙ subsequent to time t1;
IV) from one or more VCO₂ values of step III), obtaining respective FPsₜₙ with algorithm (F):
$$FPs = 4.7893 . ln [ 0.4304 . {( {ΦCO}_{2 ( e )} - 1.005 . {ΦCO}_{2 ( e )}^{0.9703} )}^{2} + 11.285 . ( {ΦCO}_{2 ( e )} - 1.005 . {ΦCO}_{2 ( e )}^{0.9703} ) + 3.3912 ] - 9.4075 \qquad ( F )$$
and
V) comparing the one or more FPsₜₙ values according to steps III) and IV) with the respective baseline value of step I) and II) and calculating the ratio FPsₜₙ/FPsₜ₁, wherein:
if there is the condition in which
the FPsₜₙ value compared to the baseline FPsₜ₁ decreased by 1 unit and the ratio FPsₜₙ/FPsₜ₁ is between 0.75 and 0.80,
then the subject has developed a risk of acute cardio-pulmonary disease,
wherein this method is optionally implemented by computer.

13. (canceled)

14. Media or carrier selected from magnetic tapes, magnetic disks, optical disks, magneto-optical disks, ROMs, PROMs, VCDs, DVDs or other computer-readable means, comprising a software implementing the method according to claim 1.

15. The method according to claim 3, wherein the NYHA functional classification for CHF based on the symptomatology associated with physical activity performed by the subject is shown in the following table:

| Symptoms (fatigue, palpitations, | |
|---|---|
| dyspnea, or anginal pain) and | |
| physical activity | NYHA class: |
| Asymptomatic, but with signs of | I |
| structural cardiac insult - No exercise | |
| restrictions | |
| Symptoms which appear only with | II |
| maximum effort | |
| Symptoms which already appear with | III |
| light effort | |
| Symptoms already at rest - Physical | IV |
| activity impossible | |

16. The method according to claim 3, comprising the following steps:
g) calculating the ratio
$$\frac{{{ΦHCO}_{3}^{-}}_{( FPs )}}{{ΦCO}_{2 ( FPS )}} where {{ΦHCO}_{3}^{-}}_{( FPs ) = {[ {HCO}_{3}^{-} ]}_{v} - {[ {HCO}_{3}^{-} ]}_{a} \times FPs}$$
$$and {{ΦCO}_{2}}_{( FPs ) = [ ( {PCO}_{2 v} - {PCO}_{2 a} ) \times 0.03 \times FPs}$$
and wherein the parameters [HCO₃⁻]ᵥ, [HCO₃⁻]ₐ, PCO₂ᵥ, PCO₂ₐ and FPs are obtained from a subject at rest;
h) assigning the value calculated according to step g) to an NYHA class for the disease CHF, wherein:
a value greater than 12.3 and preferably less than 12.5 indicates class I (NYHA classification);
a value between 11 and 12.3 indicates class II (NYHA classification);
a value less than 11 and greater than 9 indicates class III (NYHA classification); and
a value between 8 and 9 indicates class IV (NYHA classification).

17. The method according to claim 3, wherein the set of physiological parameters of steps a1) and a2) further comprises the proton concentration in venous blood ([H⁺]ᵥ) and the proton concentration in arterial blood GHIA the method comprising the following step:
i) assigning a subject suffering from CHF to class III (NYHA classification) when:
FPs≥5.5 L/min calculated with the set of parameters of step a1);
FPs<8.5 L/min calculated with the set of parameters of step a2);
ΦH⁺ (FPs)>18.8 nmol/min calculated with the set of parameters of step a1) with the following equation:
ΦH_{(FPs)}⁺=([H⁺]ᵥ−[H⁺]ₐ)×FPs
ΦH⁺(FPs)<154 nmol/min calculated with the set of parameters of step a2) with the following equation:
ΦH_{(FPs)}⁺=([H⁺]ᵥ−[H⁺]ₐ)×FPs;
$$\frac{{{ΦHCO}_{3}^{-}}_{( FPs )}}{{{ΦCO}_{2}}_{( FPs )}}$$
less than 11 and greater than 9 calculated with the set of parameters of step a1);
$$\frac{{{ΦHCO}_{3}^{-}}_{( FPs )}}{FPs} \leq 1.9 mmol / L$$
calculated with the set of parameters of step a1);
FPs/Qt<0.90 calculated with the set of parameters of step a1).
